(19) United States
(12) Patent Application Publication
(10) Pub. No.: US 2021/0347751 A1
(43) Pub. Date: Nov. 11, 2021

(54) METHOD FOR EFFICIENT EXTRACTION OF PLANT ANTHOCYANIDIN

(71) Applicants: Zhaoxiang GENG, Anhui (CN); Zongyong LIU, Anhui (CN)

(72) Inventors: Zhaoxiang GENG, Anhui (CN); Zongyong LIU, Anhui (CN)

(21) Appl. No.: 17/285,866

(22) PCT Filed: Oct. 12, 2019
(86) PCT No.: PCT/CN2019/110906

§ 371 Date: Apr. 15, 2021

(30) Foreign Application Priority Data
Oct. 15, 2018 (CN) 2018112472321

(51) Int. Cl. C07D 311/62

(52) U.S. Cl. CPC C07D 311/62

(57) ABSTRACT

The present invention relates to the technical field of bioengineering and provides a method for efficient extraction of plant anthocyanidin. The method comprises steps of: hydrolyzing anthocyanin; using one of sodium bicarbonate or potassium bicarbonate NaHCO₃/KHCO₃ to neutralize an aqueous solution obtained after the anthocyanin hydrolysis to pH 7; using a saturated aqueous solution of sodium chloride or potassium chloride NaCl/Kcl to perform salting out for suspended separation of a conjugate of an anthocyanidin sodium salt or potassium salt and a fatty acid or fatty acid ester; allowing aqueous hydrochloric acid to react with the anthocyanidin sodium salt or potassium salt; and filtering and separating an anthocyanidin aqueous solution. The present invention is applied to the extraction of natural plant anthocyanidin.

### BACKGROUND OF THE INVENTION

### 1. Field of the Invention

The present invention relates to the technical field of bioengineering and provides a method for efficient extraction of plant anthocyanidin.

### 2. Description of the Related Art

Separation and extraction of natural plant anthocyanidin has always been a problem for those skilled in the art across the world. Efficient methods for separation and extraction of natural plant anthocyanidin are not available now. In the prior art, such a method is disclosed in a Chinese Patent Application No. CN 201210516999.6, filed on Nov. 25, 2012, and publication No. CN103834702A, published on Jun. 4, 2014, entitled “METHOD FOR EXTRACTION OF CATIONIC ANTHOCYANIDIN AGLYCONE”, wherein the method comprises steps of: reacting with cationic anthocyanidins under acidic conditions (PH≤3.5) by using an anonic surfactant; based on the prior art, only when an aqueous solution is under the strong acidic condition of pH 3.5 can the chlorine cl atom on the oxygen atom on the middle C₃ ring in the anthocyanidin molecular structure (C₆—C₃—C₆) can dissociate a chloride ion cl−, so that anthocyanidins are cationic; since anthocyanidins are situated under the strong acidic condition (PH 3.5), the anionic surfactant will firstly or simultaneously react with hydrogen ion H⁺ in the acid aqueous solution to synthesize water-insoluble alkyl acid/fatty acid, however, great loss of the anionic surfactant may incur; a salt compound, formed by the synthesis of the anionic surfactant and cationic (cl⁻ is dissociated in the anthocyanidin molecule) anthocyanidins, can be dissolved in an acidic aqueous solution due to the fact that the anthocyanidin molecule structure (e.g., cyanidin) has 5 hydroxyl groups, so that the salt compound formed by the synthesis of anthocyanidins and the anionic surfactant is acid soluble, and it has some difficulty separating anthocyanidins from a large amount of water; as a result, most of the anthocyanidins hydrolyzed by β-glucosidase are still dissolved in the acid hydrolyzed aqueous solution; worse still, the salt compound formed by the synthesis of anthocyanidins and the anionic surfactant is also ionized in the acidic hydrolyzed aqueous solution, with the result that anionic groups in the anionic surfactant are separated from the anthocyanidin molecule, and anthocyanidins containing 5 hydroxyl groups cannot be extracted; through the above experiment, it proves that the method disclosed in the above-mentioned patent application can only be used to extract anthocyanidins containing 4 hydroxyl groups (e.g., peonidin), and it is such a waste of valuable anthocyanidin resources; yield of the extracted anthocyanidins is severely restricted.

In the above-mentioned patent application technology, since the anthocyanidin active aglycone is only slightly soluble in an aqueous hydrochloric acid solution (solubility is about 0.1%), hydrochloric acid is reacted with a salt compound formed by the synthesis of the anionic surfactant and the anthocyanidin active aglycone, and more than 95% of the anthocyanidin active aglycone is entrapped in the waste residue of fatty acid or alkyl (benzene) acid and cannot be separated. In addition, about 0.1-0.2% fatty acid or alkyl (benzene) acid is dissolved in the aqueous hydrochloric acid solution containing a small amount of anthocyanidin active aglycone; the aqueous hydrochloric acid solution of the anthocyanidin active aglycon is too acidic (it cannot be neutralized with alkali); people cannot eat it directly. The aqueous hydrochloric acid solution containing a small amount of the anthocyanidin active aglycone cannot be heated and dehydrated, otherwise the molecular structure of the anthocyanidin active aglycone will be destroyed. However, dehydration of the aqueous hydrochloric acid solution through vacuum freezing (sublimation) has extremely low production efficiency, involves too much energy consumption, and it takes a long period of time to dehydrate; therefore, it can only be adapted to produce the aqueous hydrochloric acid solution in laboratory on a small scale, and it is not suitable for use in industrial production.

The biological activity of anthocyanidins containing 4 hydroxyl groups is lower than that of anthocyanidins containing 5 hydroxyl groups.

Therefore, it is necessary to make substantial improvements, innovations and fundamental breakthroughs to the extraction method of anthocyanidins.

### SUMMARY OF THE INVENTION

Principle of the invention:

1. Anthocyanidins are aglycones in anthocyanins and do not contain glycosyls. Anthocyanidins cannot be reacted with strong alkali sodium hydroxide or potassium hydroxide NaOH/KOH aqueous solutions with a pH value greater than pH 7.5, otherwise the molecular structure of anthocyanidins will change and become colorless chalcone. The relative density of sodium chloride is 2.165, and the relative density of potassium chloride is 1.98. Relative density unit: gram/cubic centimeter or milliliter, which also applies to the following description.

2. In the anthocyanidin molecule, the hydrogen atoms on the hydroxyl groups originally bound by glycosidic bonds dissociate water-soluble hydrogen H⁺ ions in an aqueous solution with a pH of 4 to 7.5, and the hydrogen atoms are water-soluble anionic groups. Natural plants do not contain free anthocyanidin molecules, but exist in the form of glycosides of anthocyanidins, that is, anthocyanins. The aqueous solution of anthocyanins is hydrolyzed by β-glucosidase at a temperature of 40-50 ° C. with a pH of 4.5-5 to generate sugar and free anthocyanidin molecules.

When an acidic aqueous solution obtained after the anthocyanin hydrolysis is neutralized by using sodium bicarbonate or potassium bicarbonate NaHCO₃/KHCO₃, sodium bicarbonate or potassium bicarbonate NaHCO₃/KHCO₃ is also reacted with anthocyanidins to generate anthocyanidin sodium salts or potassium salts, and to release carbon dioxide CO₂.

3. Anthocyanidins of the present invention refer to aglycones in the glycosides of anthocyanidins (that is, in the anthocyanin), which do not contain glycosyl, that is, the active aglycon.

4. Anthocyanidins are slightly soluble in acidic aqueous solutions. Anthocyanidins produce colorless chalcone in an alkaline aqueous solution with a pH above 7.5, and the structure changes.

5. In a flavonoid structure of the anthocyanidin molecular structure (C₆—C₃—C₆), the chlorine cl atom on the oxygen O atom on the middle C₃ ring can only dissociate chlorine cl⁻ ions when the pH value of the aqueous solution is less than or equal to pH 3.5; it cannot dissociate chlorine cl⁻ ions at pH 4 of 7.5.

6. The principle of salting out of anthocyanidins/anthocyanidin sodium or potassium salts and separation of anthocyanins/glucose aqueous solutions: in the enzymatic hydrolysis of anthocyanin aqueous solution by β-glucosidase, anthocyanins cannot be completely hydrolyzed to form anthocyanidins, and it is necessary to separate anthocyanins/glucose from anthocyanidins, and to separate anthocyanidins from water, wherein the mass of water is 1000 times that of anthocyanidins.

Anthocyanins contain highly water-soluble glycosyl (e.g., glucosyl, galactosyl), and 4-6 hydrophilic hydroxyl groups (—OH) contained in the aglycone (i.e.,anthocyanidins) structure, so anthocyanins have strong hydrophilicity and water solubility; and anthocyanidins only contain 4-6 hydrophilic hydroxyl groups. Anthocyanidins available in the nature mainly refer to anthocyanidins containing 4-5 hydrophilic hydroxyl groups (—OH); the hydrophilicity of anthocyanidins is very different from that of anthocyanins, and the water solubility of anthocyanins is much higher than that of anthocyanidins.

Sodium chloride/potassium chloride Nacl/Kcl will break the hydrogen bond or polar attraction between the hydroxyl groups on anthocyanidin molecules and water molecules and reduce the water solubility of anthocyanidins and anthocyanidin sodium or potassium salts. With the increase in water solubility of sodium chloride/potassium chloride Nacl/Kcl, weakly hydrophilic anthocyanidins and anthocyanidin sodium or potassium salts will gradually separate from water and become water-insoluble substances, and the substances aggregate and precipitate, and separate from anthocyanin/glucose aqueous solutions.

The hydrophilicity of anthocyanins/glucose is greater than that of sodium chloride or potassium chloride Nacl/Kcl, and the hydrophilicity of sodium chloride or potassium chloride Nacl/Kcl is much greater than that of anthocyanidin sodium or potassium salts; glucose is more soluble in water than sodium chloride or potassium chloride Nacl/Kcl, and the hydrophilicity of glucose is stronger; so glucose and anthocyanins in water can reduce the saturated solubility of sodium chloride or potassium chloride Nacl/Kcl. Since the water solubility of anthocyanins/glucose is far greater than the hydrophilicity of anthocyanidins, it forms a great contrast; anthocyanins/glucose cannot aggregate, precipitate and separate in an aqueous solution of sodium chloride or potassium chloride Nacl/Kcl, so anthocyanidins and anthocyanidin sodium salts or potassium salts separate from an aqueous solution of anthocyanins/glucose in a saturated aqueous solution of sodium chloride or potassium chloride at a temperature of 0-50° C. with a pH of 6.8-7. The hydrophilicity of glucose is stronger than that of sodium chloride or potassium chloride Nacl/Kcl. The saturated aqueous solution of sodium chloride or potassium chloride Nacl/Kcl cannot make glucose salt out and precipitate. Anthocyanidin sodium salts or potassium salts are not soluble in neutral water or aqueous solutions of sodium chloride/potassium chloride.

The temperature of an aqueous solution of anthocyanins, obtained after the anthocyanin hydrolysis, and containing anthocyanidins and anthocyanidin sodium salts or potassium salts, is between 0° C. and 50° C., and the aqueous solution is kept neutral; salting out the aqueous solution of anthocyanins, obtained after the anthocyanin hydrolysis, and containing anthocyanidins and anthocyanidin sodium salts or potassium salts by using the saturated aqueous solution of sodium chloride or potassium chloride Nacl/Kcl, so that anthocyanidins and anthocyanidin sodium salts or potassium salts can separate from the aqueous solution obtained after the anthocyanin hydrolysis. For the saturated solubility of Nacl/Kcl in the aqueous solution obtained after the anthocyanin hydrolysis at 0° C-50° C., anthocyanidins and anthocyanidin sodium salts or potassium salts can salting out for separating from the aqueous solution obtained after the anthocyanin hydrolysis in an optimum way.

7. The relative density of impurities in oil-soluble fatty acids or fatty acid esters (oleic acid esters, palmitic acid esters) in natural plants is less than 1 (water is 1, g/cm3 or ml). Freezing point of oleic acid (V): 13.2, melting point of palmitic acid (° C).:63.1. Natural anthocyanin-containing plants (or fruits) mainly contain palmitic acid and oleic acid esters (e.g., black rice), wherein the acid ester of palmitic acid accounts for the maximum amount of the plant, and the content of the acid ester of palmitic acid are at least 5-10 times that of the anthocyanidins obtained after the anthocyanin hydrolysis.

The present invention uses palmitic acid or/and oleic acid esters to suspend and separate anthocyanidin sodium or potassium salts from an aqueous solution of anthocyanins/glucose.

Palmitic acid's scientific name is hexadecanoic acid. It is the most common fatty acid in the nature. The palmitic acid of varying amounts can be found in almost all the greases. Its molecular formula: C₁₆H₃₂O₂. its chemical formula: CH₃(CH₂)₁₄COOH. Palmitic acid is white and pearly scales, and exists in many oils and greases in the form of glycerides. It is insoluble in water, slightly soluble in cold alcohol and petroleum ether, soluble in hot ethanol, ether and chloroform, etc. Density (g/mL, 25/4° C.):0.8527; relative density (25° C., 4° C.):0.841480.

Oleic acid, together with other fatty acids, can be found in all animal and vegetable oils in the form of glycerides. Relative density (g/mL, 20/4 ° C).:0.8935, relative density (g/mL, 25/4 ° C).:0.845590, relative density (g/mL, 90/4° C.):0.8429.

8. Anthocyanidins and anthocyanidin sodium or potassium salts are insoluble in a saturated aqueous solution of sodium chloride or potassium chloride Nacl/Kcl in an aqueous solution of anthocyanins/glucose.

9. The main composition of a conjugate of anthocyanidin sodium salts and fatty acids or fatty acid esters is fatty oils or fatty acid esters (palmitic acid or/and oleic acid esters), so the relative density of the conjugate is below 1.1; while the relative density of a saline saturated aqueous solution containing anthocyanins/glucose and sodium chloride or potassium chloride NaCl/Kcl is greater than 1.1, for example, 1.2 or greater than 1.2; the conjugate of anthocyanidin sodium salts and fatty acids or fatty acid esters can be suspended and separated from the saturated aqueous solution containing anthocyanins/glucose and sodium chloride or potassium chloride NaCl/Kcl. Anthocyanidins and anthocyanidin sodium or potassium salts are insoluble in neutral water or aqueous solutions of sodium chloride/potassium chloride Nacl/Kcl.

10. The principle of enabling 10%-30% (by weight) of aqueous hydrochloric acid solution to have a double decomposition reaction with anthocyanidin sodium salts or potassium salts by stirring for recycling anthocyanidins through precipitation of the aqueous hydrochloric acid solution is as follows: shear stirring is carried out at a speed of 300-500 rpm, wherein a mass-to-liquid ratio of the anthocyanidin sodium salt or potassium salt to the aqueous hydrochloric acid Hcl solution is in a range of 10 to 20; it aims to continuously change the shape and size of particles of the anthocyanidin sodium salts or potassium salts, so that the anthocyanidin sodium salts or potassium salts can fully contact and be reacted with the aqueous hydrochloric acid Hcl solution to produce an aqueous hydrochloric acid solution of hydrogen H reduced anthocyanidins and sodium chloride or potassium chloride Nacl/Kcl. Since the anthocyanidins are only slightly soluble in the aqueous hydrochloric acid Hcl solution, the water solubility of the hydrochloric acid and sodium chloride or potassium chloride is much higher than that of the anthocyanidins, the hydrochloric acid and sodium chloride or potassium chloride also inhibit anthocyanidins dissolving in the aqueous hydrochloric acid Hcl solution. Sodium chloride or potassium chloride cannot react with anthocyanidins, since weak acid (hydroxyl groups in anthocyanidin molecules) anthocyanidin cannot generate strong acid hydrochloric acid; the reaction can be done completely. An absolute excess of anthocyanidins aggregate, precipitate and separate only due to the fact that the relative density (1.8) of anthocyanidins far more greater than that of hydrochloric acid and sodium chloride or potassium chloride.

11. Based on the prior art, L-cysteine is a reducing agent, and its role in the hydrolysis of anthocyanins is to prevent the active center of β-glucosidase from being oxidized and to restore the activity of β-glucosidase; but L-cysteine itself is oxidized into L-cystine of a disulfide bond, L-cystine is slightly soluble in water, soluble in dilute acid aqueous solution, isoelectric point (PI) of L-cystine is 4.6, and the pH value of the acid aqueous solution obtained after the anthocyanin hydrolysis to the isoelectric point (PI) of L-cystine, so that L-cystine can be precipitated and separated. The β-glucosidase enzymatic hydrolysis products of anthocyanins are anthocyanidins and glucose, but glucose has an aldehyde group, which has oxidizing and reducing properties and can oxidize the active center of β-glucosidase, so glucose is a typical inhibitor of β-glucosidase enzymatic hydrolysis; therefore, the reducing agent L-cysteine is added to prevent the active center of β-glucosidase from being oxidized and to restore the activity of β-glucosidase.

Cystine is also known as L-cystine, 3,3′-dithiodialanine, etc. Its CAS number is 56-89-3, and its chemical formula is C₅H₁₂N₂O₄S₂. It was discovered by Wollaston from bladder stones in 1810. In 1832, Berzelius named it cystine. It is a sulfur-containing amino acid, which is contained in a small amount in protein, and is mainly found in keratins in hair, fingers and paws. It can also be obtained by synthetic methods. It is applied in medicine, food, cosmetics and other industries.

L-cystine, water solubility: 0.112 g/L (25° C.), white flaky crystal or crystalline powder; solubility in water: very slightly soluble; solubility in other solvents: insoluble in ethanol and other organic solvents, easily soluble in dilute acid; isoelectric point pI4.6.

12. Sodium ascorbate (sodium vitamin C) is obtained by successively adding sodium bicarbonate and isopropanol to a solution of ascorbic acid (vitamin C), and by reacting the solution of ascorbic acid (vitamin C) and sodium bicarbonate and isopropanol. It can also be obtained by heating and filtering ascorbic acid and sodium carbonate. Sodium ascorbate and ascorbic acid have the same physiological functions (effects). They can be taken orally or injected into the blood. Anthocyanidins are taken orally by the human body and absorbed into blood through the human small intestine. Anthocyanidins are naturally non-toxic, easily degraded in the human body, and have a bioavailability of 100%. Anthocyanins have no obvious biological activity and can only be used as pigments, and their bioavailability in the human body is almost zero.

13. Activated carbon can adsorb anthocyanins in the anthocyanin aqueous solution, so it has a decolorizing effect.

14. Water solubility of sodium chloride Nacl (gram/100 g of water): 0° C.:35.7 g, 10° C.:35.8 g, 20° C.:36.0 g, 30° C.:36.3 g, 40° C.:36.6 g, 50° C.:37.0 g, 60° C.:37.3 g, 70° C.:37.8g, 80° C.:38.4 g, 90° C.:39.0 g, 100° C.:39.8 g.

15. Water solubility of potassium chloride Kcl (gram/100 g of water): 0° C. 10° C. 20° C. 30° C. 40° C. 60° C. 70° C. 80° C. 90° C. 100° C. 28g 31.2 g 34.2 g 37.2 g 40.1 g 45.8 g 48.8 g 51.3 g 53.9 g 56.3 g.

The technical solutions are as follows:

1.

1). hydrolyzing a natural plant anthocyanin aqueous solution with β-glucosidase, preventing oxidation by using L-cysteine, protecting the activity of β-glucosidase; wherein the hydrolysis is carried out at a temperature of 45-50° C. for 24-48 hours with pH of 4.5-5; an aqueous solution containing anthocyanidins/anthocyanins/glucose is obtained, and the aqueous solution contains oil-soluble fatty acid or fatty acid impurities which are dispersed in an acidic aqueous solution and have a large total surface area and an absorption surface area and have a relative density of less than 1; the fatty acid or fatty acid impurities refer to oleic acid or/and palmitic acid esters, an appropriate amount of oleic acid or/and palmitic acid esters are added to an anthocyanin aqueous solution with low oleic acid or/and palmitic acid content to perform suspended separation of an anthocyanidin sodium salts or potassium salts in an anthocyanin/glucose aqueous solution in the following step 3);

2) using sodium bicarbonate or potassium bicarbonate NaHCO₃/KHCO₃ to neutralize an aqueous solution obtained after the anthocyanin hydrolysis to pH 6.8-7, and allowing anthocyanidins to produce sodium salt or potassium salts thereof;

3) adding sodium chloride or potassium chloride NaCl/Kcl to the above-mentioned 2) to formulate a saline saturated aqueous solution of an aqueous solution containing anthocyanidin sodium salts or potassium salts/anthocyanins/glucose in step 2); the formulation method is as follows: in order to save energy, when the temperature is in the range of 25-50° C., the saturated solubility of sodium chloride or potassium chloride Nacl/Kcl in water at a certain temperature is used as a reference value, and sodium chloride or potassium chloride Nacl/Kcl at the reference value is added to and is dissolved in the aqueous solution containing anthocyanidins/anthocyanins/glucose, allowing the aqueous solution to stand for 1 to 3 minutes, to precipitate and separate the excess sodium chloride or potassium chloride immediately; an aqueous solution containing anthocyanidin sodium salts or potassium salts/anthocyanins/glucose is taken out and allowed to sit for 3 to 8 hours, such that the fatty acid or fatty acid esters entrap, absorb or physically bind to a water-insoluble conjugate of anthocyanidin sodium salts or potassium salts, and the conjugate is a solid water-insoluble matter due to its relative density is less than the relative density of a saturated aqueous solution of sodium chloride or potassium chloride NaCl/Kcl, then a densely suspended separation of the conjugate is achieved; the conjugate is then separated from the aqueous solution of anthocyanins and glucose and sodium chloride or potassium chloride; then a conjugate of anthocyanidin sodium salts or potassium salts and a fatty acid or fatty acid esters is taken out in layers, the moisture in the conjugate is filtered, and water-soluble anthocyanin, glucose and liquid oleic acid esters are also filtered; finally, a conjugate of an anthocyanidin sodium salt or potassium salt and palmitic acid ester is obtained;

4) allowing slightly excess aqueous hydrochloric acid to react with the anthocyanidin sodium salt or potassium salt in palmitic acid ester in step 3) by stirring at room temperature, and strong acid generates weak acid, and an aqueous hydrochloric acid solution of hydrogen H reduced anthocyanidins and sodium chloride or potassium chloride is produced, and anthocyanidins are completely dissolved in the aqueous hydrochloric acid solution; the palmitic acid ester is insoluble in the aqueous hydrochloric acid Hcl solution, and it is not reacted with the aqueoushydrochloric acid Hcl solution; sodium chloride or potassium chloride does not react with the aqueous hydrochloric acid Hcl solution; sodium chloride or potassium chloride does not react with the anthocyanidin,weak acid anthocyanidin cannot generate strong acid hydrochloric acid; the anthocyanidin-containing aqueous hydrochloric acid Hcl solution is more acidic, and the pH value of the aqueous solution is below 2; people are not allowed to take it directly; the present invention selects and utilizes important properties that the suspending agent palmitic acid or/and oleic acid ester is insoluble in and does not react with the aqueous hydrochloric acid Hcl solution, the anthocyanidin sodium salt or potassium salt is reduced to an aqueous hydrochloric acid solution of anthocyanidins, otherwise it makes no sense to enable the aqueous hydrochloric acid Hcl solution to react with the anthocyanidin sodium salt or potassium salts in the palmitic acid esters in step 3) as mentioned above by stirring;

5) filtering the aqueous hydrochloric acid solution of anthocyanidins containing the palmitic acid esters in the above step 4) with a compact acid-resistant industrial filter cloth under pressure or vacuum decompression at room temperature to separate the palmitic acid ester; since the palmitic acid ester is a palmitic acid solid, an amorphous substance, it can not be filtered with a filter screen; the hydrochloric acid Hcl solution of anthocyanidin is obtained; the aqueous hydrochloric acid aqueous solution containing anthocyanidins is neutralized by using an aqueous solution of sodium bicarbonate or potassium bicarbonate NaHCO₃/KHCO₃, so that its acidic pH is adjusted to 3.5-4; when the aqueous solution containing the anthocyanidin is neutralized by using the aqueous solution of sodium bicarbonate or potassium bicarbonate NaHCO₃/KHCO₃, the aqueous solution of sodium bicarbonate or potassium bicarbonate NaHCO₃/KHCO₃ must be added slowly for several times and it must be stirred quickly at a stirring speed of 300-500 rpm, so that sodium bicarbonate or potassium bicarbonate is evenly distributed in the aqueous hydrochloric acid solution containing anthocyanidins; an appropriate amount of ascorbic acid (i.e., vitamin C) is added to the aqueous hydrochloric acid solution containing anthocyanidins and having a pH of 3.5-4, after the pH value of the aqueous hydrochloric acid solution containing anthocyanidins is adjusted to pH 1-2, the aqueous hydrochloric acid solution containing anthocyanidins and having a pH of 1-2 is neutralized to pH of 3.4-4 by using the aqueous solution of sodium bicarbonate or potassium bicarbonate NaHCO₃/KHCO₃; hydrochloric acid is a strong acid, and ascorbic acid (etc., vitamin C) is a weak acid; here, the competitive reaction of ascorbic acid (etc., vitamin C) with hydrochloric acid greatly reduces the amount of hydrochloric acid in the hydrochloric acid Hcl aqueous solution of anthocyanidins, so that most hydrochloric acid is replaced with ascorbic acid (etc., vitamin C); even if there is a small amount of water-soluble ascorbic acid, that is, the sodium or potassium salt of vitamin C, human body can consume the substance; anthocyanidins are slightly dissolved in an acidic aqueous solution; red ascorbic acid of anthocyanins, namely vitamin C aqueous solution, is obtained;

6) if there is a small amount of anthocyanidin sodium salt or potassium salt products in the above step (5), allowing the ascorbic acid containing anthocyanidins above, that is, the vitamin C aqueous solution, to stand at room temperature for 24 to 72 hours, so that the small amount of anthocyanidin sodium salt or potassium salt products are aggregated, precipitated and separated, or filtered with a compact acid-resistant industrial filter cloth under pressure or vacuum decompression; the small amount of anthocyanidin sodium salt or potassium salt products are reacted with the aqueous hydrochloric acid Hcl solution for repeated uses; red ascorbic acid of anthocyanin, namely vitamin C aqueous solution, is obtained.

2. A method for producing solid samples for the detection of the anthocyanidin products according to the present invention comprises steps of:

(1) taking out the conjugate of an anthocyanidin sodium salt or potassium salt and a fatty acid or fatty acid ester in step 3) in the above-mentioned technical solution 1, filtering the moisture in the conjugate, wherein water-soluble anthocyanin and glucose are also filtered; then edible soft water is added to the conjugate of an anthocyanidin sodium salt or potassium salt and the fatty acid or fatty acid ester, wherein a volume of the edible soft water is 10 to 20 times that of the conjugate, and water consumption is greatly reduced when compared with the aqueous solution obtained after the anthocyanin hydrolysis; an appropriate amount of sodium chloride or potassium chloride NaCl/Kcl is added at a temperature of 0-5 ° C. to reach a concentration of a saturated aqueous solution thereof, so that the relative density of the aqueous solution is greater than 1; then the aqueous solution stands at a temperature of 0-5° C. for 8-24 hours, so as to enable solid particles of the fatty acid or fatty acid ester to collide ad aggregate, and to greatly reduce a total surface area and an absorption surface area of the fatty acid or fatty acid ester, and a densely suspended separation of the fatty acid or fatty acid ester in a form of fatty solid is achieved, and solid particles of the anthocyanidin sodium salt or potassium salt also collide, aggregate, precipitate and separate from each other; the relative density of anthocyanidin sodium salt or potassium salt is greater than 18, which is far more greater than the relative density of the saturated aqueous solution of sodium chloride or potassium chloride NaCl/Kcl;

2) filtering and taking out the anthocyanidin sodium salts or potassium salts in layers in the above-mentioned step 1), making the anthocyanidin sodium salts or potassium salts reach with an excess of 10-30% (by weight) aqueous hydrochloric acid Hcl solution under continuous stirring operation, wherein the stirring speed is 300-500 rpm, so that the anthocyanidin sodium salts or potassium salts are fully reacted with the aqueous hydrochloric acid Hcl solution, and an aqueous solution of hydrogen-reduced anthocyanidin precipitate and sodium chloride or potassium chloride is produced; a mass-to-liquid ratio of the anthocyanidin sodium salts or potassium salts to the aqueous hydrochloric acid Hcl solution is in a range of 10 to 20; the anthocyanidin precipitate is removed by filtering or chromatography technique, hydrochloric acid in the anthocyanidin precipitate is washed with a small amount of edible water at a temperature of 1-25° C., to precipitate ad separate anthocyanidins, and an anthocyanidin solid is obtained; then the anthocyanidin solid is subjected to vacuum freeze dehydrating and drying, so as to obtain an anthocyanidin solid product having the same chemical structure as natural anthocyanidins; the aqueous hydrochloric acid Hcl solution containing a small amount of anthocyanidins are recycled for further use; the relative density of the anthocyanidin is 1.8.

In the technical solution 1 of the present invention, many steps are provided, that is, the anthocyanins are hydrolyzed, using sodium bicarbonate or potassium bicarbonate NaHCO₃/KHCO₃ to neutralize an aqueous solution obtained after the anthocyanin hydrolysis to pH 7, using a saturated aqueous solution of sodium chloride or potassium chloride NaCl/Kcl to perform salting out for suspended separation of anthocyanidin sodium salts or potassium salts, allowing hydrochloric acid to react with the anthocyanidin sodium salts or potassium salts, filtering step, using one of sodium bicarbonate or potassium bicarbonate NaHCO₃/KHCO₃ to neutralize and reduce the acidity of hydrochloric acid, and hydrochloric acid is replaced with ascorbic acid (etc., vitamin C), all of those steps are core technologies of the present invention, that is, technical features necessary for the inventive steps.

By adopting the above-mentioned technical solutions, the present invention has the following beneficial effects.

1. The productivity of anthocyanidins is greatly improved on an industrial scale, and all anthocyanidins, subjected to hydrolysis with β-glucosidase, can be extracted, wherein anthocyanidins here include anthocyanidins containing 4 hydroxyl groups, anthocyanidins containing 5 hydroxyl groups, and anthocyanidins containing 6 hydroxyl groups.

2. Waste of anthocyanidin resource is avoided.

3. Only a small amount of water needs to be dried, so energy is greatly saved and the production efficiency of anthocyanidins on an industrial scale is greatly improved.

4. The anthocyanidin aqueous solution product according to the present invention can be consumed directly by users, and it is convenient to use, and it is completely and quickly absorbed by the human body.

5. An aqueous solution of anthocyanidins and acid according to the present invention only contains anthocyanidins, a trace amount of hydrochloric acid, vitamin C (and its sodium or potassium salts), and sodium chloride or potassium chloride; the anthocyanidin purity is high and it can contain water-insoluble chemical compositions and other harmful chemical compositions, so it can be used as a human blood injection.

Preferred technical solutions of the present invention are as follows:

1). hydrolyzing a natural black rice anthocyanin aqueous solution with β-glucosidase, preventing oxidation by using L-cysteine, protecting the activity of β-glucosidase; wherein the hydrolysis is carried out at a temperature of 50° C. for 48 hours with pH of 4.5; an aqueous solution containing anthocyanidins/anthocyanins/glucose is obtained, and the aqueous solution contains oil-soluble fatty acid or fatty acid impurities which are dispersed in an acidic aqueous solution and have a large total surface area and an absorption surface area and have a relative density of less than 1; the fatty acid or fatty acid impurities refer to oleic acid or/and palmitic acid esters, an appropriate amount of oleic acid or/and palmitic acid esters are added to an anthocyanin aqueous solution with low oleic acid or/and palmitic acid content to perform suspended separation of an anthocyanidin sodium salt in an anthocyanin aqueous solution in the following step 3);

2) using sodium bicarbonate NaHCO₃ to neutralize an aqueous solution obtained after the anthocyanin hydrolysis to 6.8-7, and allowing anthocyanidins to produce sodium salts thereof;

3) adding sodium chloride NaCl to step 2) to formulate saline saturated aqueous solution of an aqueous solution containing anthocyanidin sodium salts/anthocyanins/glucose; the formulation method is as follows: in order to save energy, when the temperature is in the range of 25-50° C., the saturated solubility of sodium chloride Nacl in water at a certain temperature is used as a reference value, and sodium chloride Nacl at the reference value is added to and is dissolved in the aqueous solution containing anthocyanidin/anthocyanin/glucose, allowing the aqueous solution to stand for 1 to 3 minutes, to precipitate and separate the excess sodium chloride or potassium chloride immediately; an aqueous solution containing anthocyanidin sodium salts/anthocyanins/glucose is taken out and allowed to sit for 8 hours, such that the fatty acid or fatty acid esters entrap, absorb or physically bind to a water-insoluble conjugate of an anthocyanidin sodium salt, and the conjugate is a solid water-insoluble matter due to its relative density is less than the relative density of a saturated aqueous solution of sodium chloride NaCl, then a densely suspended separation of the conjugate is achieved; the conjugate is then separated from the aqueous solution of anthocyanin and glucose and sodium chloride; then a conjugate of an anthocyanidin sodium salt and a fatty acid or fatty acid ester is taken out in layers, the moisture in the conjugate is filtered, and water-soluble anthocyanin, glucose and liquid oleic acid esters are also filtered; finally, a conjugate of an anthocyanidin sodium salt and palmitic acid ester;

4) allowing slightly excess aqueous hydrochloric acid solution to react with the anthocyanidin sodium salt in palmitic acid ester in step 3) by stirring at room temperature, and strong acid generates weak acid, and an aqueous hydrochloric acid solution of hydrogen H reduced anthocyanidin and sodium chloride is produced, and the anthocyanidin is completely dissolved in the aqueous hydrochloric acid solution; the palmitic acid ester is insoluble in the aqueous hydrochloric acid Hcl solution, and it is not reacted with the aqueous hydrochloric acid Hcl solution; sodium chloride does not react with the aqueous hydrochloric acid Hcl solution; sodium chloride does not react with the anthocyanidin, weak acid anthocyanidin cannot generate strong acid hydrochloric acid; the anthocyanidin-containing aqueous hydrochloric acid Hcl solution is more acidic, and the pH value of the aqueous solution is below 2; people are not allowed to take it directly; the present invention selects and utilizes important properties that the suspending agent palmitic acid or/and oleic acid ester is insoluble in and does not react with the aqueous hydrochloric acid Hcl solution, the anthocyanidin sodium salt is reduced to an aqueous hydrochloric acid solution of anthocyanidin, otherwise it makes no sense to enable the aqueous hydrochloric acid Hcl solution to react with the anthocyanidin sodium salt in the palmitic acid ester in step 3) as mentioned above by stirring;

5) filtering the aqueous hydrochloric acid solution of anthocyanidins containing the palmitic acid ester in the above step 4) with a compact acid-resistant industrial filter cloth under pressure or vacuum decompression at room temperature to separate the palmitic acid ester; since the palmitic acid ester is a palmitic acid solid, an amorphous substance, it can not be filtered with a filter screen; the aqueous hydrochloric acid Hcl solution of anthocyanidin is obtained; the aqueous hydrochloric acid solution containing anthocyanidins is neutralized by using an aqueous solution of sodium bicarbonate NaHCO₃, so that its acidic pH is adjusted to 3.5-4; when the aqueous solution containing anthocyanidins is neutralized by using the aqueous solution of sodium bicarbonate NaHCO₃, the aqueous solution of sodium bicarbonate NaHCO₃ must be added slowly for several times and it must be stirred quickly at a stirring speed of 300-500 rpm, so that sodium bicarbonate is evenly distributed in the aqueous hydrochloric acid solution containing anthocyanidins; an appropriate amount of ascorbic acid (i.e., vitamin C) is added to the aqueous hydrochloric acid solution containing anthocyanidins and having a pH of 3.5-4, after the pH value of the aqueous hydrochloric acid solution containing anthocyanidins is adjusted to pH 1-2, the aqueous hydrochloric acid solution containing anthocyanidins and having a pH of 1-2 is neutralized to pH of 3.5-4 by using the aqueous solution of sodium bicarbonate NaHCO₃; hydrochloric acid is a strong acid, and ascorbic acid (etc., vitamin C) is a weak acid; here, the competitive reaction of ascorbic acid (etc., vitamin C) with hydrochloric acid greatly reduces the amount of hydrochloric acid in the aqueous hydrochloric acid Hcl solution of anthocyanidins, so that most hydrochloric acid is replaced with ascorbic acid (etc., vitamin C); even if there is a small amount of water-soluble ascorbic acid, that is, the sodium or potassium salt of vitamin C, human body can consume the substance; anthocyanidin is slightly dissolved in an acidic aqueous solution; red ascorbic acid of anthocyanins, namely vitamin C aqueous solution, is obtained;

6) if there is a small amount of anthocyanidin sodium salt products in the above step (5), allowing the ascorbic acid containing the anthocyanidins above, that is, the vitamin C aqueous solution, to stand at room temperature for 72 hours, so that the small amount of anthocyanidin sodium salt products are aggregated, precipitated and separated or filtered with a compact acid-resistant industrial filter cloth under pressure or vacuum decompression; the small amount of anthocyanidin sodium salt products are reacted with the aqueous hydrochloric acid Hcl solution for repeated uses; red ascorbic acid of anthocyanins, namely vitamin C aqueous solution, is obtained.

### DETAILED DESCRIPTION

### Example 1

1). Hydrolyzing a natural black rice aqueous anthocyanin solution with β-glucosidase, preventing oxidation by using L-cysteine, protecting the activity of β-glucosidase; wherein the hydrolysis is carried out at a temperature of 45-50° C. for 24-48 hours with pH of 4.5-5; an aqueous solution containing anthocyanidins/anthocyanins/glucose is obtained, and the aqueous solution contains oil-soluble fatty acid or fatty acid impurities which are dispersed in an acidic aqueous solution and have a large total surface area and an absorption surface area and have a relative density of less than 1; the fatty acid or fatty acid impurities refer to oleic acid or/and palmitic acid esters, an appropriate amount of oleic acid or/and palmitic acid esters are added to an anthocyanin aqueous solution with low oleic acid or/and palmitic acid content to perform suspended separation of an anthocyanidin sodium salt or potassium salt in an anthocyanin/glucose aqueous solution in the following step 3);

2) using sodium bicarbonate NaHCO₃ to neutralize an aqueous solution obtained after the anthocyanin hydrolysis to 6.8-7, and allowing anthocyanidins to produce sodium salt or potassium salts thereof;

3) adding sodium chloride NaCl to the above-mentioned 2) to formulate saline saturated aqueous solution of an aqueous solution containing anthocyanidin sodium salt or potassium salts/anthocyanins/glucose in step 2); the formulation method is as follows: in order to save energy, when the temperature is in the range of 25-50° C., the saturated solubility of sodium chloride Nacl in water at a certain temperature is used as a reference value, and sodium chloride Nacl at the reference value is added to and is dissolved in the aqueous solution containing anthocyanidins/anthocyanins/glucose, allowing the aqueous solution to stand for 1 to 3 minutes, to precipitate and separate the excess sodium chloride immediately; an aqueous solution containing anthocyanidin sodium salt/anthocyanin/glucose is taken out and allowed to sit for 3 to 8 hours, such that the fatty acid or fatty acid esters entrap, absorb or physically bind to a water-insoluble conjugate of an anthocyanidin sodium salt, and the conjugate is a solid water-insoluble matter due to its relative density is less than the relative density of a saturated aqueous solution of sodium chloride NaCl, then a densely suspended separation of the conjugate is achieved; the conjugate is then separated from the aqueous solution of anthocyanin and glucose and sodium chloride; then a conjugate of an anthocyanidin sodium salt and a fatty acid or fatty acid ester is taken out in layers, the moisture in the conjugate is filtered, and water-soluble anthocyanins, glucose and liquid oleic acid esters are also filtered; finally, a conjugate of an anthocyanidin sodium salt and palmitic acid ester;

4) allowing slightly excess aqueous hydrochloric acid solution to react with the anthocyanidin sodium salt or potassium salt in palmitic acid ester in step 3) by stirring at room temperature, and strong acid generates weak acid, and an aqueous hydrochloric acid solution of hydrogen H reduced anthocyanidin and sodium chloride is produced, and the anthocyanidin is completely dissolved in the aqueous hydrochloric acid solution; the palmitic acid ester is insoluble in the aqueous hydrochloric acid Hcl solution, and it is not reacted with the aqueous hydrochloric acid Hcl solution; sodium chloride does not react with the aqueous hydrochloric acid Hcl solution; sodium chloride does not react with anthocyanidins, weak acid anthocyanidin cannot generate strong acid hydrochloric acid; the anthocyanidin-containing aqueous hydrochloric acid Hcl solution is more acidic, and the pH value of the aqueous solution is below 2; people are not allowed to take it directly; the present invention selects and utilizes important properties that the suspending agent palmitic acid or/and oleic acid ester is insoluble in and does not react with the aqueous hydrochloric acid Hcl solution, the anthocyanidin sodium salt is reduced to an aqueous hydrochloric acid solution of anthocyanidins, otherwise it makes no sense to enable the aqueous hydrochloric acid Hcl solution to react with the anthocyanidin sodium salt in the palmitic acid ester in step 3) as mentioned above by stirring;

5) filtering the aqueous hydrochloric acid solution of anthocyanidins containing the palmitic acid ester in the above 4) with a compact acid-resistant industrial filter cloth under pressure or vacuum decompression at room temperature to separate the palmitic acid ester; since the palmitic acid ester is a palmitic acid solid, an amorphous substance, it can not be filtered with a filter screen; the aqueous hydrochloric acid Hcl solution of anthocyanidins is obtained; the aqueous hydrochloric acid solution containing anthocyanidins is neutralized by using an aqueous solution of sodium bicarbonate NaHCO₃, so that its acidic pH is adjusted to 3.5-4; when the aqueous solution containing anthocyanidins is neutralized by using the aqueous solution of sodium bicarbonate NaHCO₃, the aqueous solution of sodium bicarbonate NaHCO₃ must be added slowly for several times and it must be stirred quickly at a stirring speed of 300-500 rpm, so that sodium bicarbonate is evenly distributed in the aqueous hydrochloric acid solution containing anthocyanidins; an appropriate amount of ascorbic acid (i.e., vitamin C) is added to the aqueous hydrochloric acid solution containing anthocyanidins and having a pH of 3.5-4, after the pH value of the aqueous hydrochloric acid solution containing anthocyanidins is adjusted to pH 1-2, the aqueous hydrochloric acid solution containing anthocyanidins and having a pH of 1-2 is neutralized to pH of 3.4-4 by using the aqueous solution of sodium bicarbonate NaHCO₃; hydrochloric acid is a strong acid, and ascorbic acid (etc., vitamin C) is a weak acid; here, the competitive reaction of ascorbic acid (etc., vitamin C) with hydrochloric acid greatly reduces the amount of hydrochloric acid in the aqueous hydrochloric acid Hcl solution of anthocyanidins, so that most hydrochloric acid is replaced with ascorbic acid (etc., vitamin C); even if there is a small amount of water-soluble ascorbic acid, that is, the sodium or potassium salt of vitamin C, human body can consume the substance; anthocyanidins are slightly dissolved in an acidic aqueous solution; red ascorbic acid of anthocyanin, namely vitamin C aqueous solution, is obtained;

6) if there is a small amount of anthocyanidin sodium salt products in the above step (5), allowing the ascorbic acid containing the anthocyanidins above, that is, the vitamin C aqueous solution, to stand at room temperature for 24 hours, so that the small amount of anthocyanidin sodium salt products are aggregated, precipitated and separated, or filtered with a compact acid-resistant industrial filter cloth under pressure or vacuum decompression; the small amount of anthocyanidin sodium salt products are reacted with the aqueous hydrochloric acid Hcl solution for repeated uses; red ascorbic acid of anthocyanin, namely vitamin C aqueous solution, is obtained.

### Example 2

1). Hydrolyzing a natural plant anthocyanin aqueous solution with β-glucosidase, preventing oxidation by using L-cysteine, protecting the activity of β-glucosidase; wherein the hydrolysis is carried out at a temperature of 45-50° C. for 24-48 hours with pH of 4.5-5; an aqueous solution containing anthocyanidins/anthocyanins/glucose is obtained, and the aqueous solution contains oil-soluble fatty acid or fatty acid impurities which are dispersed in an acidic aqueous solution and have a large total surface area and an absorption surface area and have a relative density of less than 1; the fatty acid or fatty acid impurities refer to oleic acid or/and palmitic acid esters, an appropriate amount of oleic acid or/and palmitic acid esters are added to an anthocyanin aqueous solution with low oleic acid or/and palmitic acid content to perform suspended separation of an anthocyanidin sodium salt or potassium salt in an anthocyanin/glucose aqueous solution in the following step 3);

2) using potassium bicarbonate KHCO₃to neutralize an aqueous solution obtained after the anthocyanin hydrolysis in the step 1) to 6.8-7, and allowing the anthocyanidins to produce potassium salts thereof;

3) adding potassium chloride Kcl to the above-mentioned 2) to formulate saline saturated aqueous solution of an aqueous solution containing anthocyanidin potassium salts/anthocyanins/glucose in step 2); the formulation method is as follows: in order to save energy, when the temperature is in the range of 25-50° C., the saturated solubility of potassium chloride Kcl in water at a certain temperature is used as a reference value, and potassium chloride Kcl at the reference value is added to and is dissolved in the aqueous solution containing anthocyanidin potassium salts/anthocyanins/glucose, allowing the aqueous solution to stand for 1 to 3 minutes, to precipitate and separate the excess potassium chloride immediately; an aqueous solution containing anthocyanidin sodium salts or potassium salts/anthocyanins/glucose is taken out and allowed to sit for 3 hours, such that the fatty acid or fatty acid esters entrap, absorb or physically bind to a water-insoluble conjugate of an anthocyanidin potassium salt, and the conjugate is a solid water-insoluble matter due to its relative density is less than the relative density of a saturated aqueous solution of potassium chloride Kcl, then a densely suspended separation of the conjugate is achieved; the conjugate is then separated from the aqueous solution of anthocyanins and glucose and potassium chloride; then a conjugate of an anthocyanidin potassium salt and a fatty acid or fatty acid ester is taken out in layers, the moisture in the conjugate is filtered, and water-soluble anthocyanin, glucose and liquid oleic acid esters are also filtered; finally, a conjugate of an anthocyanidin potassium salt and palmitic acid ester;

4) allowing slightly excess aqueous hydrochloric acid solution to react with the anthocyanidin potassium salt in palmitic acid ester in step 3) by stirring at room temperature, and strong acid generates weak acid, and a hydrochloric acid aqueous solution of hydrogen H reduced anthocyanidins and potassium chloride is produced, and the anthocyanidin is completely dissolved in the aqueous hydrochloric acid solution; the palmitic acid ester is insoluble in the aqueous hydrochloric acid Hcl solution, and it is not reacted with the hydrochloric acid Hcl aqueous potassium chloride does not react with the aqueous hydrochloric acid Hcl solution; potassium chloride does not react with the anthocyanidin, weak acid anthocyanidin cannot generate strong acid hydrochloric acid; the anthocyanidin-containing aqueous hydrochloric acid Hcl solution is more acidic, and the pH value of the aqueous solution is below 2; people are not allowed to take it directly; the present invention selects and utilizes important properties that the suspending agent palmitic acid or/and oleic acid ester is insoluble in and does not react with the aqueous hydrochloric acid Hcl solution, the anthocyanidin potassium salt is reduced to an aqueous hydrochloric acid solution of anthocyanidins, otherwise it makes no sense to enable the aqueous hydrochloric acid Hcl solution to react with the anthocyanidin potassium salt in the palmitic acid ester in step 3) as mentioned above by stirring;

5) filtering the hydrochloric acid aqueous solution of anthocyanidins containing the palmitic acid ester in the above 4) with a compact acid-resistant industrial filter cloth under pressure or vacuum decompression at room temperature to separate the palmitic acid ester; since the palmitic acid ester is a palmitic acid solid, an amorphous substance, it can not be filtered with a filter screen; the aqueous hydrochloric acid Hcl solution of anthocyanidin is obtained; the aqueous hydrochloric acid solution containing the anthocyanidin is neutralized by using an aqueous solution of potassium bicarbonate KHCO₃, so that its acidic pH is adjusted to 3.5-4; when the aqueous solution containing anthocyanidins is neutralized by using the aqueous solution of potassium bicarbonate KHCO₃, the aqueous solution of potassium bicarbonate KHCO₃ must be added slowly for several times and it must be stirred quickly at a stirring speed of 300-500 rpm, so that potassium bicarbonate is evenly distributed in the aqueous hydrochloric acid solution containing anthocyanidins; an appropriate amount of ascorbic acid (i.e., vitamin C) is added to the aqueous hydrochloric acid solution containing the anthocyanidin and having a pH of 3.5-4, after the pH value of the aqueous hydrochloric acid solution containing anthocyanidins is adjusted to pH 1-2, the aqueous hydrochloric acid solution containing anthocyanidins and having a pH of 1-2 is neutralized to pH of 3.4-4 by using the aqueous solution of potassium bicarbonate KHCO₃; hydrochloric acid is a strong acid, and ascorbic acid (etc., vitamin C) is a weak acid; here, the competitive reaction of ascorbic acid (etc., vitamin C) with hydrochloric acid greatly reduces the amount of hydrochloric acid in the aqueous hydrochloric acid Hcl solution of anthocyanidins, so that most hydrochloric acid is replaced with ascorbic acid (etc., vitamin C); even if there is a small amount of water-soluble ascorbic acid, that is, potassium salt of vitamin C, human body can consume the substance; anthocyanidins are slightly dissolved in an acidic aqueous solution; red ascorbic acid of anthocyanins, namely vitamin C aqueous solution, is obtained;

6) if there is a small amount of anthocyanidin potassium salt products in the above step (5), allowing the ascorbic acid containing anthocyanidins above, that is, the vitamin C aqueous solution, to stand at room temperature for 72 hours, so that the small amount of anthocyanidin potassium salt products are aggregated, precipitated and separated, or filtered with a compact acid-resistant industrial filter cloth under pressure or vacuum decompression; the small amount of potassium salt products are reacted with the aqueous hydrochloric acid Hcl solution for repeated uses; red ascorbic acid of anthocyanin, namely vitamin C aqueous solution, is obtained.

Each specific numerical value in the data range of the present invention can be implemented according to the principle of the invention, and the data range includes each specific numerical value therein, which can be independently selected and implemented by those skilled in the art according to the principle of the invention. In order to save the length of the description, each specific numerical value in the data range will not be repeated in the present invention. Reactions are all conducted at room temperature under atmospheric conditions, unless otherwise stated in the present invention.

What is claimed is:

1. A method for efficient extraction of plant anthocyanidin, comprising:
1). hydrolyzing a natural plant anthocyanin aqueous solution with β-glucosidase, preventing oxidation by using L-cysteine, protecting the activity of β-glucosidase; wherein the hydrolysis is carried out at a temperature of 45-50° C. for 24-48 hours with pH of 4.5-5;an aqueous solution containing anthocyanidins/anthocyanins/glucose is obtained, and the aqueous solution contains oil-soluble fatty acid or fatty acid impurities which are dispersed in an acidic aqueous solution and have a large total surface area and an absorption surface area and have a relative density of less than 1; the fatty acid or fatty acid impurities refer to oleic acid or/and palmitic acid esters, an appropriate amount of oleic acid or/and palmitic acid esters are added to an anthocyanin aqueous solution with low oleic acid or/and palmitic acid content to perform suspended separation of an anthocyanidin sodium salt or potassium salt in an anthocyanin/glucose aqueous solution in the following process 3);
2) using one of sodium bicarbonate or potassium bicarbonate NaHCO₃/KHCO₃ to neutralize the pH of an aqueous anthocyanin solution obtained in step 1) to 6.8-7, and allowing anthocyanidins to produce sodium salts or potassium salts thereof;
3) adding the above 2) to sodium chloride or potassium chloride NaCl/Kcl to formulate saline saturated aqueous solution of an aqueous solution containing anthocyanidin sodium salts or potassium salts/anthocyanins/glucose in 2); the formulation method is as follows: in order to save energy, when the temperature is in the range of 25-50° C., the saturated solubility of sodium chloride or potassium chloride Nacl/Kcl in water at a certain temperature is used as a reference value, and sodium chloride or potassium chloride Nacl/Kcl at the reference value is added to and is dissolved in the aqueous solution containing anthocyanidins/anthocyanins/glucose, allowing the aqueous solution to stand for 1 to 3 minutes, to precipitate and separate the excess sodium chloride or potassium chloride immediately; an aqueous solution containing anthocyanidin sodium salts or potassium salts/anthocyanins/glucose is taken out and allowed to sit for 3 to 8 hours, such that the fatty acid or fatty acid esters entrap, absorb or physically bind to a water-insoluble conjugate of an anthocyanidin sodium salt or potassium salt, and the conjugate is a solid water-insoluble matter due to its relative density is less than the relative density of a saturated aqueous solution of sodium chloride or potassium chloride NaCl/Kcl, then a densely suspended separation of the conjugate is achieved; the conjugate is then separated from the aqueous solution of anthocyanins and glucose and sodium chloride or potassium chloride; then a conjugate of an anthocyanidin sodium salt or potassium salt and a fatty acid or fatty acid ester is taken out in layers, the moisture in the conjugate is filtered, and water-soluble anthocyanin, glucose and liquid oleic acid esters are also filtered; finally, a conjugate of an anthocyanidin sodium salt or potassium salt and palmitic acid ester;
4) allowing slightly excess aqueous hydrochloric acid to react with the anthocyanidin sodium salt or potassium salt in palmitic acid ester in step 3) by stirring at room temperature, and strong acid generates weak acid, and an aqueous hydrochloric acid solution of hydrogen H reduced anthocyanidin and sodium chloride or potassium chloride is produced, and anthocyanidins are completely dissolved in the aqueous hydrochloric acid solution; the palmitic acid ester is insoluble in the aqueous hydrochloric acid Hcl solution, and it is not reacted with the aqueous hydrochloric acid Hcl solution; sodium chloride or potassium chloride does not react with the aqueous hydrochloric acid Hcl solution; sodium chloride or potassium chloride does not react with the anthocyanidin,weak acid anthocyanidin cannot generate strong acid hydrochloric acid; the anthocyanidin-containing aqueous hydrochloric acid Hcl solution is more acidic, and the pH value of the aqueous solution is below 2; people are not allowed to take it directly; the present invention selects and utilizes important properties that the suspending agent palmitic acid or/and oleic acid ester is insoluble in and does not react with the aqueous hydrochloric acid Hcl solution, the anthocyanidin sodium salt or potassium salt is reduced to an aqueous hydrochloric acid solution of anthocyanidin;
5) filtering the aqueous hydrochloric acid solution of anthocyanidins containing the palmitic acid ester in the above 4) with a compact acid-resistant industrial filter cloth under pressure or vacuum decompression at room temperature to separate the palmitic acid ester; since the palmitic acid ester is a palmitic acid solid, an amorphous substance, it can not be filtered with a filter screen; the aqueous hydrochloric acid Hcl solution of anthocyanidins is obtained; the aqueous hydrochloric acid solution containing anthocyanidins is neutralized by using an aqueous solution of sodium bicarbonate or potassium bicarbonate NaHCO₃/KHCO₃, so that its acidic pH is adjusted to 3.5-4;
when the aqueous solution containing anthocyanidins is neutralized by using the aqueous solution of sodium bicarbonate or potassium bicarbonate NaHCO₃/KHCO₃, the aqueous solution of sodium bicarbonate or potassium bicarbonate NaHCO₃/KHCO₃ must be added slowly for several times and it must be stirred quickly at a stirring speed of 300-500 rpm, so that sodium bicarbonate or potassium bicarbonate is evenly distributed in the aqueous hydrochloric acid solution containing anthocyanidins; an appropriate amount of ascorbic acid (i.e., vitamin C) is added to the aqueous hydrochloric acid solution containing anthocyanidins and having a pH of 3.5-4, after the pH value of the aqueous hydrochloric acid solution containing anthocyanidins is adjusted to pH 1-2, the aqueous hydrochloric acid solution containing anthocyanidins and having a pH of 1-2 is neutralized to pH of 3.4-4 by using the aqueous solution of sodium bicarbonate or potassium bicarbonate NaHCO₃/KHCO₃; hydrochloric acid is a strong acid, and ascorbic acid (etc., vitamin C) is a weak acid; here, the competitive reaction of ascorbic acid (etc., vitamin C) with hydrochloric acid greatly reduces the amount of hydrochloric acid in the aqueous hydrochloric acid Hcl solution of anthocyanidins, so that most hydrochloric acid is replaced with ascorbic acid (etc., vitamin C); even if there is a small amount of water-soluble ascorbic acid, that is, the sodium or potassium salt of vitamin C, human body can consume the substance; anthocyanidins are slightly dissolved in an acidic aqueous solution; red ascorbic acid of anthocyanin, namely vitamin C aqueous solution, is obtained;
6) if there is a small amount of anthocyanidin sodium salt or potassium salt products in the above step (5), allowing the ascorbic acid containing anthocyanidins above, that is, the vitamin C aqueous solution, to stand at room temperature for 24 to 72 hours, so that the small amount of anthocyanidin sodium salt or potassium salt products are aggregates, precipitates and separates, or filtered with a compact acid-resistant industrial filter cloth under pressure or vacuum decompression; the small amount of anthocyanidin sodium salt or potassium salt products are reacted with the aqueous hydrochloric acid Hcl solution for repeated uses;
red ascorbic acid of anthocyanin, namely vitamin C aqueous solution, is obtained; reactions are all conducted at room temperature under atmospheric conditions, unless otherwise stated in the present invention.

2. The method for efficient extraction of plant anthocyanidin of claim 1, wherein a method for producing solid samples for the detection of the anthocyanidin products according to the present invention comprises steps of:
(1) taking out the conjugate of an anthocyanidin sodium salt or potassium salt and a fatty acid or fatty acid ester in step 3) in the above-mentioned technical solution 1, filtering the moisture in the conjugate, wherein water-soluble anthocyanins and glucose are also filtered; then edible soft water is added to the conjugate of an anthocyanidin sodium salt or potassium salt and the fatty acid or fatty acid ester, wherein a volume of the edible soft water is 10 to 20 times that of the conjugate, and water consumption is greatly reduced when compared with the aqueous solution obtained after the anthocyanin hydrolysis; an appropriate amount of sodium chloride or potassium chloride NaCl/Kcl is added at a temperature of 0-5° C. to reach a concentration of a saturated aqueous solution thereof, so that the relative density of the aqueous solution is greater than 1; then the aqueous solution stands at a temperature of 0-5° C. for 8-24 hours, so as to enable solid particles of the fatty acid or fatty acid ester to collide ad aggregate, and to greatly reduce a total surface area and an absorption surface area of the fatty acid or fatty acid ester, and a densely suspended separation of the fatty acid or fatty acid ester in a form of fatty solid is achieved, and solid particles of the anthocyanidin sodium salt or potassium salts also collide, aggregate, precipitate and separate from each other; the relative density of anthocyanidin sodium salts or potassium salts is greater than 18, which is far more greater than the relative density of the saturated aqueous solution of sodium chloride or potassium chloride NaCl/Kcl;
2) filtering and taking out the anthocyanidin sodium salts or potassium salts in layers in the above-mentioned step 1), making the anthocyanidin sodium salts or potassium salts reach with an excess of 10-30% (by weight) aqueous hydrochloric acid Hcl solution under continuous stirring operation, wherein the stirring speed is 300-500 rpm, so that the anthocyanidin sodium salts or potassium salts are fully reacted with the aqueous hydrochloric acid Hcl solution, and an aqueous solution of hydrogen-reduced anthocyanidin precipitate and sodium chloride or potassium chloride is produced; a mass-to-liquid ratio of the anthocyanidin sodium salt or potassium salt to the aqueous hydrochloric acid Hcl solution is in a range of 10 to 20; the anthocyanidin precipitate is removed by filtering or chromatography technique, hydrochloric acid in the anthocyanidin precipitate is washed with a small amount of edible water at a temperature of 1-25° C., to precipitate ad separate the anthocyanidin, and an anthocyanidin solid is obtained; then the anthocyanidin solid is subjected to vacuum freeze dehydrating and drying, so as to obtain an anthocyanidin solid product having the same chemical structure as natural anthocyanidins; the aqueous hydrochloric acid Hcl solution containing a small amount of anthocyanidins is recycled for further use; the relative density of the anthocyanidin is 1.8.
